(19) United States
(12) Patent Application Publication
(10) Pub. No.: US 2024/0277960 A1
(43) Pub. Date: Aug. 22, 2024

(54) APPARATUS FOR SUPPLYING GAS, SUCH AS NO, TO A PATIENT, WITH COUNTING OF THE TOTAL ACTUAL TREATMENT TIME

(71) Applicant: INOSYSTEMS, Antony (FR)

(72) Inventors: Yann BLANDIN, Antony (FR); Mary SCHMITT, Antony (FR); Frederic MARCHAL, Antony (FR)

(21) Appl. No.: 18/428,357

(22) Filed: Jan. 31, 2024

(30) Foreign Application Priority Data
Feb. 20, 2023 (FR) 2301524

(51) Int. Cl. A61M 16/00

(52) U.S. Cl. CPC A61M 16/024; A61M 16/0075; A61M 2202/0007; A61M 2202/0275; A61M 2205/3327; A61M 2205/3331; A61M 2205/505; A61M 2205/52

(57) ABSTRACT

The invention relates to a device (1) for supplying therapeutic gas, in particular NO/N2 mixtures, comprising an internal passage (2), valve means (5), control means (6) with microprocessor, and selection means (8), which can be actuated by a user and are configured to allow the user to start a supply of gas or stop a supply of gas. The selection means (8) supply the control means (6) with a command signal corresponding to the choice made by the user, in order to control, via the valve means (5), the flow of gas in the internal passage (2), i.e. to authorize or stop any flow of gas. The control means (6) count a total duration of actual supply of gas equal to the sum of all the time periods elapsing between a choice, made by the user, of starting a supply of gas and a choice, made by the user, of stopping a supply of gas. Installation for administering gas to a patient, which includes such a gas supply apparatus.

### CROSS-REFERENCE TO RELATED APPLICATIONS

This patent application claims priority to FR2301524, filed Feb. 20, 2023, and the entire contents of which are incorporated herein by reference.

### FIELD OF ART

The present invention relates to an apparatus or device for supplying therapeutic gas, in particular nitric oxide (NO), and furthermore to an installation for administering gas to a patient, comprising such an apparatus for supplying therapeutic gas. The apparatus includes means for counting and recording the actual treatment time of all the patients who have been treated by gas administration through the apparatus since the time of the first use of the apparatus, excluding pauses or periods of non-use of the apparatus, for example between two consecutive patients.

### BACKGROUND

Inhaled nitric oxide, or NOi, is a gaseous medicament commonly used to treat patients suffering from acute pulmonary arterial hypertension, in particular pulmonary vasoconstrictions in adults or children, including the newborn, or PPHN (persistent pulmonary hypertension of the newborn), as described for example by EP-A-560928 or EP-A-1516639.

An installation for implementation of a treatment by NOi, commonly known as an NO administration installation, conventionally comprises one or more cylinders of NO/N2 mixture which feed an NO administration device and/or treatment monitoring device, a medical ventilator for delivering a respiratory gas, such as an O2/N2 mixture or air, to which is added the NO (i.e. NO/N2), and a patient kit comprising in particular a respiratory interface, such as an endotracheal tube, and one or more flexible conduits.

An NO administration installation of this kind is used in a hospital environment for administering NOi treatment and thus for treating hospital patients who need to inhale NO in order to treat their pulmonary arterial hypertension.

However, the dose regimen for the treatment of the patients varies from one patient to another, namely the dosage prescribed by the doctor, the mode of ventilation and the minute volume set on the medical ventilator and the duration of administration (given that it may be continuous or discontinuous when a resumption of NO administration is required after a pause or a failed attempt at withdrawal.

Therefore, within hospitals, it is very difficult to trace the use made of this medicament by healthcare teams and to follow the customary consumption data.

A similar problem may also exist for other therapeutic gases, in particular equimolar mixtures of oxygen and nitrous oxide (MEOPA), i.e. O2/N2O mixtures.

However, knowing the duration of treatment and therefore of actual use of the apparatuses is necessary for various reasons, such as medical reasons relating to the treatment to be followed by the patient, reasons relating to maintenance of the apparatus, accounting reasons, in particular invoicing, reasons relating to proper management of the use of the apparatuses in a hospital structure, reasons relating to monitoring the consumption of gas, or other reasons.

Some gas supply devices, in particular for NO, incorporate a counter which is triggered and starts counting the treatment time as soon as it detects a change in position (i.e. closed or open) of the valve controlling the gas outlet of a pressurized-gas cylinder, such as a cylinder containing an NO/N2 gas mixture, which feeds the gas supply device in question. However, such a system is not ideal because the apparatus does not know how to recognize and take into account the pauses in the gas treatment or the test periods or the like. These can last from a few minutes to several hours, which means that the counter continues to count these pause times as treatment times as long as the valve is not closed by the user. The treatment time counted is therefore necessarily inaccurate and imprecise.

EP-A-3821929 proposes counting the total number of patients treated by administration of therapeutic gas, such as NO, in response to the user pressing a key of the gas supply apparatus at the commencement of a gas administration, and then again at the end of the gas administration. This makes it possible to know and display the number of patients who have inhaled the gas and the precise duration of the actual treatment of each patient, i.e. short or long treatment. However, the total duration of use of the apparatus, during which gas is administered to the patient, is not determined by the apparatus, nor is it displayed.

Medical personnel, such as doctors, nurses or others, are sometimes asked directly to record in writing, typically on a sheet of paper or in a notebook, all the available data concerning the start of treatment, any adjustment of treatment (e.g. ventilation, dosage, etc.) and any possible pause or attempted stopping of treatment. Calculations then need to be made to determine the total duration of use of the gas supply apparatus. It is easy to see how this manner of proceeding leads to errors during transcription and calculation, to omissions, to loss of written data, etc., and it is therefore not a suitable solution.

A problem is therefore to be able to know precisely and automatically the actual treatment time during which the apparatus has supplied a therapeutic gas which has been used to effectively treat a patient by inhalation of said therapeutic gas, in particular NO, which actual treatment time is available for consultation at any moment by the user, such as the healthcare staff, in order to provide useful information to the user in an instant, i.e. in (quasi) real time, without the healthcare staff having to perform manual recording or any calculation of the duration.

### SUMMARY

One solution of the invention thus relates to a gas supply apparatus or device for supplying or delivering a therapeutic gas to a patient, in particular a gas containing NO, such as an NO/N2 mixture, comprising:
at least one internal passage for supplying a flow of gas, comprising valve means,
control means (or control device) with microprocessor (i.e. one or more microprocessors) instructing the valve means to control the supply of gas, that is to say to authorize or stop/prevent the circulation of gas in said at least one internal passage, and
selection means (or selection device) which can be actuated by a user and are configured to allow the user to start or stop a supply of gas.

According to the invention, the control means are configured to count a total duration (Dtot) of actual supply of gas by the apparatus corresponding to the cumulative total (i.e. the algebraic sum) of all the time periods (dti) that have elapsed between a start and a stop of a supply of gas by the apparatus, that is to say over the course of time, to the successive patients who have received gas as part of their treatment, i.e. to all the patients who have been treated successively by administration of gas using the apparatus.

In other words, the total duration (Dtot) of actual supply of gas, as counted according to the invention, corresponds to the sum or cumulative total of all the periods or times of actual use (dti) of the apparatus for actually treating patients, from the first use of the apparatus, that is to say from the first patient treated, excluding all the periods of non-supply of gas to the patient. All the periods of non-supply of gas to the patient include the periods of non-use of the apparatus, such as between two successive patients, and the periods in which the apparatus is used but without supplying gas intended to treat the patient, for example a pause time of the apparatus during the treatment of a given patient, a maintenance operation or a functional test, a demonstration of operation or the like.

The total duration (Dtot) of actual supply of gas is therefore such that:

Dtot=Σdt(I) with: i≥1

where: i is the number of periods dt of actual use of the gas considered, i.e. of all the patients treated, hence typically i>1.

The total duration (Dtot) of actual supply of gas is therefore equal to a cumulative total (i.e. addition or sum) of all the periods of actual use of the apparatus for treating patients, and this since its first use (i.e. with a first patient) or commissioning (i.e. the total and actual time of administration of gas to the patients), but excludes all the periods of non-use of the apparatus, in particular the one or more pause periods that have taken place during the treatment of at least one patient or the periods of non-use separating two successive uses for two consecutive patients, or others. Thus, the periods of maintenance of the apparatus, during which ‘test gas’ may be supplied, are not counted either.

There are various ways of excluding these maintenance periods or others periods, for example excluding durations below a given minimum duration threshold (e.g. 10, 15 or 20 min), or those that elapse after the operator presses a specific key (e.g. test initiation) or after connection, to the apparatus to be tested/maintained, of a specific maintenance device (e.g. a USB key) or others.

Nevertheless, most of these periods of non-use have a negligible duration, i.e. of a few minutes in general, compared to the periods of normal and effective use of the apparatus, i.e. during therapeutic treatment of a patient with administration of gas, in particular inhaled NO, which typically last several hours or tens of hours, including from one to several days.

Therefore, counting (i.e. not excluding) maintenance/test periods and the like is not desirable but would not distort, or would distort in a very negligible and/or non-representative manner, the calculation of the total duration (Dtot) of actual supply of gas, in particular over long periods, for example over several weeks or a fortiori over several consecutive months.

Depending on the embodiment considered, the therapeutic gas supply apparatus or device of the invention can comprise one or more of the following features:
it further comprises selection means which can be actuated by a user and are configured to allow the user to choose between starting a supply of gas and stopping a supply of gas, i.e. to start or stop/interrupt a supply of gas.
the total duration (Dtot) of actual supply of gas by the apparatus corresponds to the cumulative total (i.e. algebraic sum) of all the time periods (dti) that have elapsed between a choice, made by the user, of starting a supply of gas (DF) and a choice, made by the user, of stopping a supply of gas (AF), by actuating (e.g. finger pressure or other selection mode) the selection means of the apparatus.
said at least one internal passage is configured to convey the gas flow between (at least) one gas inlet and (at least) one gas outlet supplying the gas.
it comprises several internal gas passages, in particular a main line and a bypass line, equipped with valve means.
the selection means cooperate with the control means to supply them with at least one command signal corresponding to the choice made by the user (i.e. starting or stopping the supply of gas).
the control means (or control device) instruct the valve means (or valve device) to control the flow of gas in the internal passage, in response to the command signal supplied by the selection means, so as to authorize or stop (i.e. prohibit or interrupt) any flow of gas in said internal passage.
the control means are configured to control a display, on the graphical display, of the total duration (Dtot) of actual supply of gas.
the control means are configured to control a display, on the graphical display, of one or more time periods (dti) of one or more patients.
an algorithm comprises or acts as (i.e. serves as) a time counter, that is to say that the time counting is performed by a duration-counting algorithm.
the duration-counting algorithm, that is to say the time counter, is implemented by the control means.
the time counter is configured to count all the time periods (dti) elapsing between a choice, made by the user, of starting a supply of gas and a choice, made by the user, of stopping a supply of gas, excluding any period of non-supply of gas (and preferably the other periods mentioned above: test, maintenance).
the choice of starting a supply of gas corresponds to a start of treatment of a given patient, including commencement of a supply of gas, or to a resumption of treatment that has been interrupted in this given patient, in particular in the event of the supply of gas being paused.
the choice of stopping a supply of gas corresponds to a permanent cessation of treatment of said given patient, including a permanent cessation of supply of gas to this patient, or a temporary cessation of a treatment that has started in a given patient, in particular when the supply of gas to said patient is paused.
it comprises storage means, also called a storage device, for storing the total duration (Dtot) of actual supply of gas and/or all the time periods (dti).
the time counter, or timer, is integrated into the control means, in particular into a microprocessor of the control means.
the time counter is configured to start counting the total duration (Dtot) of actual supply of gas in response to a choice, made by the user, of starting a supply of gas by actuation of the selection means, that is to say when initiating treatment of the patient at the start of a treatment of said patient or upon a resumption (re-start) of a treatment that has been paused.
the control means are configured to initiate or stop the time counter in response to the command signal delivered by the selection means, in particular a gas supply start or stop signal, respectively.
the time counter is configured to interrupt the counting of the total duration (Dtot) of actual supply of gas in response to a choice of stopping a supply of gas, i.e. following a temporary stop (i.e. pause) or a permanent stop of treatment of a patient.
the time counter is configured not to count time during the periods of non-use of the apparatus, i.e. when the apparatus does not supply gas, in particular any pause period and/or any period between two successive treatments and/or any maintenance/test period or any other period of non-supply of gas.
the total duration (Dtot) of actual supply of gas counted by the counter is updated automatically as soon as the counter starts, i.e. during each period of supply of gas.
the counter is configured (for all the patients treated successively):
i) to count the total duration (Dtot) of supply of gas only during actual supply of gas to a given patient, i.e. when the apparatus delivers gas intended for this patient, and
(ii) to stop all counting of total duration (Dtot) as soon as the actual supply of gas to this given patient is interrupted, whether this interruption is permanent or temporary, for example during the time of resetting the apparatus between two successive patients, during a pause in the course of treatment of a patient, during a phase of testing or calibrating the apparatus, during a demonstration phase, etc.
the counter is configured to repeat steps (i) and (ii) for all successive patients, i.e. patients treated successively one after another, by means of the apparatus, with actual supply of gas to each of said patients.
the time counter is configured to continue incrementing, i.e. counting, the total duration (Dtot) of actual supply of gas, at the start of supply of gas to a following patient, without resetting said time counter, after completion of actual supply of gas to a previous patient who has been treated just before the following patient. Thus, if the first patient has received gas for 9 hours 15 minutes, the time counter will start at 9 hours 15 minutes (and not at 0 hour 0 minute) when gas starts to be supplied to the next patient, i.e. the second patient, and it will then display 23 hours 45 minutes for example as the total duration (Dtot) of actual supply of gas, at the end of the supply of gas to the second patient, if the latter has received gas for 14 hours 30 minutes (i.e. =9 hours 15 minutes+14 hours 30 minutes), and so on with the following patients.
the total duration (Dtot) of actual supply of gas is expressed in days and/or hours and/or minutes.
the control means cooperate with the graphical display to display the total duration (Dtot) of actual supply of gas that has been counted.
the control means comprise at least one (micro)processor, preferably at least one microcontroller.
the one or more microprocessors are arranged on one or more electronic cards.
the control means comprise at least one microprocessor (i.e. one or more microprocessors) implementing one or more algorithms, in particular at least one calculation algorithm configured to make it possible to determine the total duration (Dtot) of actual supply of gas.
the selection means comprise one or more selection keys.
the one or more selection keys are one or more virtual keys displayed on the touch screen, i.e. one or more touch keys.
the touch screen is an interactive screen with a touch panel making it possible to display information, data or similar and also to make choices, selections, etc., by digital pressure (finger of the user) or using a stylus directly on a region of the screen, especially on virtual keys displayed by the screen.
the graphical display comprises a touch screen, and the one or more selection keys are virtual keys displayed on the touch screen.
the graphical display comprises a digital display screen, that is to say with a touch panel. The touch screen therefore comprises a touch panel.
the graphical display is configured to display the one or more virtual selection keys on the digital display screen, in the form of one or more selection regions, panes or windows or the like.
the graphical display is configured to additionally display information, measurements, curves, alerts, graphs or any other data useful to the user in the context of treatment of a patient.
the display screen is in colour or black and white, or both. For example, the user can choose a colour display or a black and white display, according to his or her preference.
the graphical display is electrically connected to the control means so as to be able to transmit, to the control means, the choices or selections made by the user via the selection means, typically one or more touch keys displayed on the touch panel display screen.
the control means are configured to instruct the valve means to effect a supply of gas in response to a selection by the user, i.e. an actuation of the selection means by the user, of a first choice corresponding to a start of supply of gas to the patient, that is to say a start of treatment of the patient, or to a resumption of a supply of gas (after a pause), that is to say a re-start of treatment of the patient in question.
conversely, the control means are configured to instruct the valve means to stop any gas supply in response to a selection by the user, i.e. to an actuation of the selection means by the user, of a second choice corresponding to a permanent cessation of supply of gas to the patient, i.e. a cessation of treatment of the patient, or a pause in the supply of gas, i.e. a temporary cessation of supply of gas to the patient, hence of the patient's treatment by administration of therapeutic gas.
it comprises a “Start” of treatment selection key or button, preferably a virtual key, commanding a start or a resumption of a supply of gas to a given patient.
it comprises a key or a button, preferably a virtual key, for “Stop” or “End” treatment, commanding a temporary or permanent stop of the supply of gas to said patient in question.
the “Start” selection key (or button) and the “Stop” (or “End”) treatment selection key (or button) are one and the same key (or button) commanding the start and the end of the supply of gas.
alternatively, according to another embodiment, the treatment “Start” key (or button) and the treatment “Stop” (or “End”) key or button are different keys (or buttons), in particular a first and a second key (or button).
the selection means can also optionally comprise one or more other selection keys (or buttons), for example:
   a “Pause” key for suspending or temporarily stopping the treatment, hence the supply of therapeutic gas, after a start of supply of gas, and/or
   a treatment “Resume” key that commands a resumption or re-start of treatment with supply of gas to a given patient after a pause period.
the “Pause” and “Resume” treatment keys (or buttons) are a single key commanding the pause and resumption of supply of gas or, alternatively, separate keys (or buttons).
it can also comprise a confirmation key (or button), preferably a virtual one displayed on the display screen, for confirming a choice that is made, for example for confirming that the patient is the same patient or another patient after stopping the supply of gas, in particular for distinguishing between a start after a temporary cessation (i.e. a pause) in the treatment of one and the same patient and after a permanent cessation of a treatment of a given patient and at the time of the start of the treatment of a new patient.
it also comprises means for storing information, data, measurements, calculations, etc., in particular computer, i.e. digital, storage means.
the storage means are configured to store (i.e. record) the total duration (Dtot) of actual supply of gas and/or all the time periods (dti).
the storage means comprise one or more computer memories for recording and storing data, for example one or more volatile or non-volatile memories, for example of the flash memory or other type.
at least one memory (e.g. flash memory) is arranged on a or the electronic card.
it also comprises electrical-current supply means (i.e. a device) which supply electrical current to those components of the gas supply apparatus that require it to operate, such as a display, microprocessor(s), memory (memories), etc. The electrical-current supply means can comprise, in a conventional manner, one or more rechargeable or non-rechargeable batteries, a power cord and an electric plug connectable to the mains network (110/220 V), a current transformer, etc.
it also comprises a rigid external casing, shell or housing in which all or some of the components of the device are arranged, in particular the control means, the internal passage, the valve means, etc.
the graphical display is arranged in or borne by a wall of the rigid external casing.
the casing is made of polymer.
the control means are configured to control (at least) the valve means so as to adjust the flow rate of therapeutic gas circulating in the internal passage.
the valve means comprise one or more solenoid valves, preferably several solenoid valves arranged in parallel.
the valve means comprise at least one proportional (solenoid) valve.
it further comprises a flowmeter device arranged on the gas passage so as to determine (i.e. measure) the flow rate of therapeutic gas circulating in the internal passage.
the flowmeter device is of the hot-wire or differential-pressure type.
the flowmeter device cooperates with the control means.
it further comprises a pressure-regulating device, such as a gas pressure reducer, arranged on the gas passage in order to control, regulate or adjust the therapeutic gas pressure within the internal passage.
it further comprises couplings and/or connectors for fluidically connecting gas lines thereto, such as one or more flexible tubes supplying or discharging gas, or for electrically connecting electric cables or the like thereto.
according to one embodiment, the control means, in particular the time counter, are also configured to determine, i.e. to measure, the instantaneous duration (Dinst) of an ongoing treatment of a given patient, that is to say the total time of treatment of this specific patient, in particular a treatment by NO.
the display screen is configured to display the instantaneous duration (Dinst), i.e. the total time of the ongoing patient treatment, in particular a treatment with NO.
the control means are configured to control a display of the instantaneous duration (Dinst) on the graphical display.
it comprises reset or re-initialization means, preferably a re-initialization key, to reset the display of the instantaneous duration (Dinst) on the graphical display. This counting and display of the instantaneous duration (Dinst) related to an ‘ongoing treatment’ are reset when a healthcare provider initiates a start of a new treatment, by clicking on a “Start” treatment key or “New patient” key.
the control means are further configured to determine the number of short treatments (TC) for which the total treatment duration is less than a predetermined threshold duration and/or the respective durations (DTC) of these short treatments (TC). Preferably, short treatments (TC) are excluded from the count, treatments having a short minimum duration, that is to say a low threshold, correspond to an exceptional situation not corresponding to a patient treatment, for example a correction of an adjustment error, a test or the like, typically a low threshold of less than or equal to 30 min, preferably less than or equal to 20 min, typically less than or equal to 10 min. In other words, the number TC corresponds to the number of patients who have undergone actual treatment but whose treatment was quickly interrupted because the treatment was not effective or had negative side effects, or for some other reason.
the control means are further configured to determine the number of long treatments (TL) for which the total treatment duration is greater than said predetermined threshold duration and/or the respective durations (DTL) of these long treatments (TL). In other words, the number TL corresponds to the number of patients who have undergone a treatment of long duration because it has been effective in these patients, for example over a duration that may be as much as several days.
typically, the predetermined threshold duration is less than or equal to about 6 hours.
the total duration (Dtot) of actual supply of gas by the apparatus thus also corresponding to the sum of the durations (DTL) of the long treatments (TL) and the durations (DTC) of the short treatments (TC), namely: Dtot=DTL+DTC

The invention also relates to an installation for administering gas to a patient, in particular for administering a gaseous mixture containing NO, comprising:
at least one source of gas, in particular of NO-based therapeutic gas, such as an NO/N2 mixture,
a gas supply apparatus according to the invention, fed with gas by said at least one gas source, and
a gas feed line fed with gas by the gas supply apparatus according to the invention.

Depending on the embodiment considered, the gas administration installation of the invention can comprise one or more of the following features:
it also comprises a medical ventilator, i.e. a ventilatory assistance apparatus, in fluidic communication with the gas feed line.
the medical ventilator is fluidically connected to the feed line in order to feed said feed line with a respiratory gas containing at least 21% by volume of oxygen, in particular air or an N2/O2 mixture.
the medical ventilator is a breathing assistance apparatus that supplies the gas at constant pressure.
alternatively, the medical ventilator is an HFO (high frequency oscillation) ventilator delivering the gas by high frequency oscillations.
the one or more gas sources contain gaseous NO, in particular a gaseous NO/N2 mixture.
the gas feed line is fed with a therapeutic gas, preferably an NO/N2 mixture, by the gas supply apparatus and with a respiratory gas containing at least 20% by volume of oxygen, preferably at least 21% by volume of O2, advantageously air or an N2/O2 mixture, by the medical ventilator.
the gas source contains an NO/N2 mixture containing less than 2000 ppm by volume of NO, the remainder being nitrogen, preferably less than 1000 ppm by volume of NO, the remainder being nitrogen.
preferably, the source of therapeutic gas contains an NO/N2 mixture containing from 250 to 900 ppm by volume of NO, the remainder being nitrogen, for example of the order of 800 ppm by volume of NO, the remainder being nitrogen.
at least one source of therapeutic gas contains an O2/N2O mixture, preferably an equimolar mixture (50%/50% mol) of oxygen and nitrous oxide, i.e. MEOPA.
it further comprises a gas humidifier arranged on the gas feed line, preferably downstream of the site where the therapeutic gas supply device is fluidically connected to said gas feed line so as to feed it with therapeutic gas.
it further comprises a line for recovering the gases exhaled by the patient.
the gas feed line and the exhaled gas recovery line are connected to a connection piece, preferably a Y-piece, and define a patient circuit.
the gas feed line forms an inspiratory branch of the patient circuit.
the exhaled gas recovery line forms an expiratory branch of the patient circuit.
the gas feed line is fluidically connected to an outlet port of the medical ventilator in order to collect and convey the gas delivered by the medical ventilator.
the exhaled gas recovery line is fluidically connected to an inlet port of the medical ventilator so as to supply the medical ventilator with all or some of the gases exhaled by the patient and thus determine any gas leaks.
the exhaled gas recovery line optionally comprises (i.e. this is not mandatory) a CO2 removal device for removing the CO2 present in the gases exhaled by the patient.
the exhaled gas recovery line optionally comprises (i.e. this is not mandatory) a filter.
at least one source of therapeutic gas comprises one or more gas containers, particularly one or more pressurized-gas cylinders.
the one or more gas containers are equipped with a gas distribution valve.
the gas distribution valve is a valve with or without integrated pressure reducer (RDI).
the gas distribution valve is made of copper alloy, such as brass.
the one ore more gas containers are equipped with a protective covering arranged around the gas distribution valve.
the covering is made of a polymer (i.e. plastic) material, of metal or of combinations of these.
the fluid container is a pressurized-gas cylinder containing, when full, a gas mixture, in particular NO/N2, at a pressure of at least 150 to 200 bar abs, or even at least 250 to 300 bar abs.
the fluid container has a generally cylindrical shape, in particular ogival.

### BRIEF DESCRIPTION OF THE VIEWS OF THE DRAWINGS

The invention will now be better understood from the following detailed description given as a non-limiting example and with reference to the appended figures, in which:

FIG. 1 shows schematically an embodiment of an apparatus for supplying therapeutic gas, in particular NO, according to the invention,

FIG. 2 shows schematically an embodiment of a gas administration installation according to the invention including the apparatus of FIG. 1,

FIG. 3 shows schematically an embodiment of a display screen of the apparatus of FIG. 1, and

FIG. 4 is an illustrative graphical representation for visualizing the total duration (Dtot) counted according to the invention.

### DETAILED DESCRIPTION

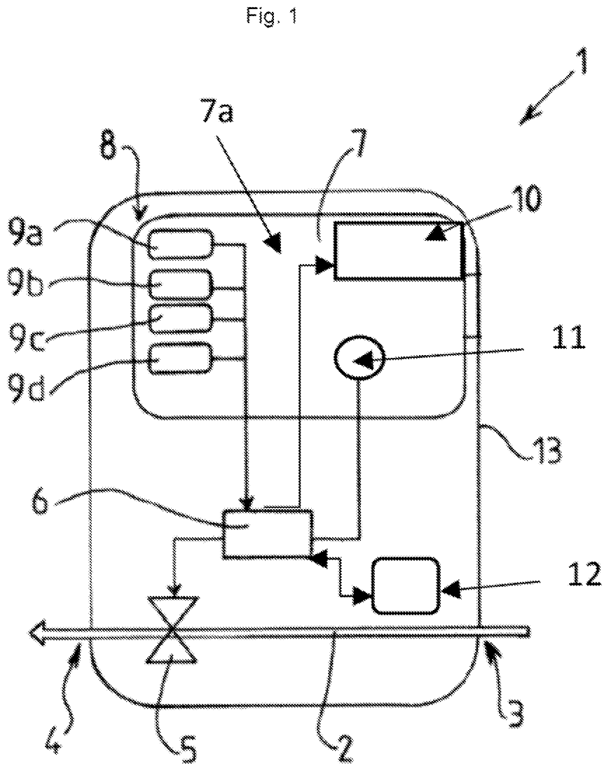

FIG. 1 shows schematically an embodiment of an apparatus or device 1 for supplying therapeutic gas according to the invention, comprising a rigid housing 13, for example made of polymer, within which is arranged an internal gas passage 2, such as a gas conduit or the like, for conveying a flow of therapeutic gas.

In this embodiment, we consider, by way of an example of a therapeutic gas, a flow of gas based on NO, i.e. typically an NO/N2 gaseous mixture formed of nitric oxide (NO) and nitrogen (N2), making it possible to treat various pulmonary pathologies, in particular pulmonary vasoconstrictions and/or pulmonary hypertension, for example persistent pulmonary hypertension of the newborn (PPHN).

The NO-based gaseous flow enters and flows through the internal gas passage 2 between one or more gas inlets 3 and one or more gas outlets 4. The gas inlets 3 and outlets 4 can be carried, for example, by mechanical and fluidic connectors or endpieces borne by the housing 13 to the apparatus 1, to which gas lines are attached that serve to convey gas as explained below, for example flexible tubes or the like.

Valve means 5, typically one or more solenoid valves, typically arranged in parallel, are arranged on the internal gas passage 2 and make it possible to control the flow of therapeutic gas which circulates in the internal passage 2, in the direction from the gas inlet 3 to the gas outlet 4, for example one or more proportional (solenoid) valves.

Optionally, a gas pressure-reducing device is arranged upstream of the valve means 5 in order to regulate the pressure of the NO-based gaseous flow within the internal gas passage 2, in particular to guarantee a given pressure, for example several bars.

The valve means 5 are controlled by control means 6 arranged in the housing 13, typically an electronic card comprising one or more microprocessors, typically one or more microcontrollers, implementing one or more algorithms. The control means 6 make it possible in particular to adjust or control the flow rate of gas passing through the valve means 5.

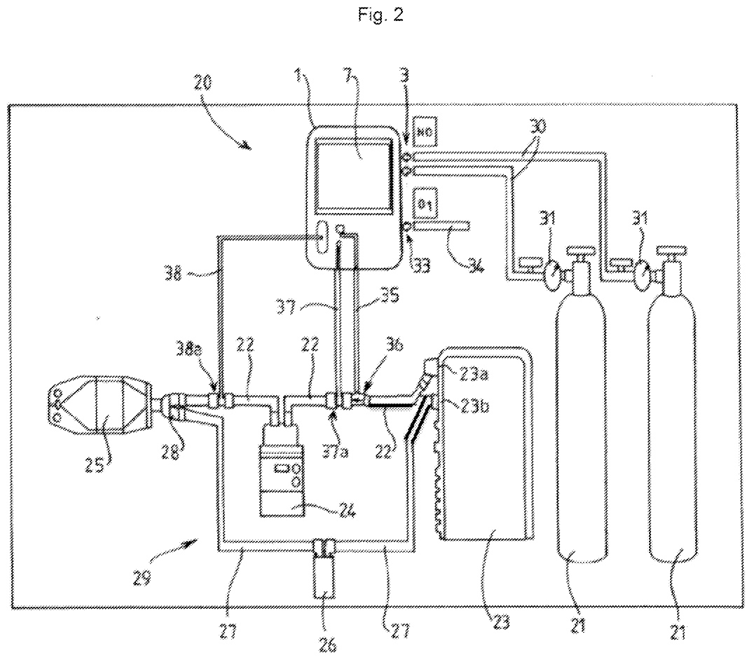

Thus, the control means 6 can control the valve means 5, such as solenoid valves arranged in parallel, that is to say can open or close all or some of these valves, in order to obtain a gas flow rate (Q) determined/calculated by the microcontroller from a value set/fixed for example by the user and as a function of the gas flow rate (Q′), i.e. air, delivered by the ventilator 23 of FIG. 2, as explained below.

One or more flowmeters (not shown) are also preferably arranged on the internal gas passage 2, upstream and/or downstream of the valve means 5 in order to determine the flow rate of NO-based gas (Q). The flowmeter can be of the differential-pressure, hot-wire or some other type. It cooperates with the control means 6 in order to supply them with measurements.

Furthermore, the housing 13 comprises a graphical display 7, preferably a touch screen 7a, that is to say with a digital panel, serving to display various items of information or data, in particular various choices selectable by a user. These choices can be displayed in windows or the like displayed on the touch/digital screen 7a of the graphical display 7.

The graphical display 7 is also configured to show one or more curves, graphs, alerts, icons, etc., or any other information or data useful to the user, such as a healthcare provider.

Also provided are selection means 8 allowing the user to make a selection among the selectable choices which are displayed on the graphical display 7, namely to choose between several options proposed and displayed on the screen, or to confirm/validate or reject a choice (i.e. an option), or else to make adjustments or settings, for example to select the desired value for the flow rate.

The selection means 8 typically comprise selection keys or buttons 9a-9d that can be actuated by the user. Advantageously, the selection means 8 comprise touch-sensitive selection keys 9a-9d, that is to say touch-sensitive keys displayed on the graphical display 7, typically a touch-sensitive screen.

Advantageously, the selection means 8, typically touch-sensitive selection keys 9a-9d, make it possible to make a choice among several possible choices. For example, in the illustrated embodiment:
a touch key 9a for starting a supply of gas, i.e. for starting treatment of a patient, i.e. a treatment “Start” key,
a touch key 9b for stopping the supply of gas, that is to say for permanently stopping the treatment of a patient, i.e. treatment “Stop” or “End” key,
a touch key 9c for pausing the supply of gas, i.e. for temporarily stopping the treatment of a patient, and for resuming the supply of gas after a pause, i.e. a treatment “Pause” and “Resume” key.
a touch key 9d for validating a choice (optional), for example for indicating whether the start of the supply of gas concerns the same patient (in the case of resumption after a pause) or, conversely, a new patient (after the end of treatment of a first patient).

As has already been indicated, according to the embodiment considered, the touch keys 9a for starting the supply of gas and 9b for stopping the supply of gas can be “merged” into a single key controlling the supply of gas and stopping the supply of gas, or they can even be merged with the touch key 9c in order also to control the pauses and the resumptions after the pauses. Depending on the desired embodiment, certain different choices can therefore be controlled by a single key, i.e. a single common key, rather than by different keys.

The graphical display 7, typically a touch-panel screen, is therefore configured to allow an operator to make the various choices mentioned above, i.e. start, pause, resumption and permanent cessation of the gas supply. For example, following the example above, the keys 9a, 9b can be “merged” into a single key for starting/stopping the supply of gas. Similarly, the keys 9c, 9d can be “merged” into a single pause/restart key.

The pressure of the user's finger on one of the virtual keys 9a-9d displayed on the touch screen 7a of the display 7, in order to make one of the aforementioned choices, will be transmitted to the microprocessor(s) of the control means 6 and the latter will then control the operation of the apparatus (i.e. supplying gas or stopping the supply of gas) as a function of the selection made and, in parallel, that of the time counter, i.e. the start or stop of the time counter in order to count or not count the time of use of the apparatus 1, as explained below.

Thus, the control means 6 can be configured to control the valve means 5, in response to the pressing of one of the virtual keys 9a-9d displayed on the touch screen 7a of the display 7, and therefore to the reception of the corresponding signal by said control means 6, in order to:
allow gas to flow in the internal passage 2 through the flow control solenoid valve(s) according to the flow rate (Q) set, in the direction of the patient if the key 9a has been pressed corresponding to the start of treatment by administration of the therapeutic gas to the patient, such as NO, i.e. the “Start of treatment” key 9a, or the key 9c for resumption after a pause;
interrupt the flow of gas in the internal passage 2 towards the patient if the key corresponding to a temporary stop (pause) or permanent cessation of treatment of the patient is pressed, that is to say the “End” of treatment key 9b or the “Pause” key 9c.

The counting, by the control means 6, of the total duration of active administration of medical gas to the patient is effected by means of the time counter or timer, typically an internal (e.g. integrated) counter of the microprocessor of the control means 6 or the like.

In other words, as soon as a treatment by administration of gas, such as NO, has to begin in a patient, that is to say at the commencement of the supply of gas, the user presses the touch-sensitive “Start” of treatment key 9a displayed on the graphical display 7 corresponding to the initiation, i.e. the start, of this treatment, which initiates the supply of gas and the counting of the treatment time by the time counter. The counter is interrupted at each pause or stop of the supply of gas.

According to the invention, the counting of the time therefore takes place only during the actual supply of gas, such as NO, to the patients and is interrupted in all situations in which the gas is not supplied to the patient, such as pauses, inter-patient stops, etc. Maintenance/test periods are also not counted.

In general, according to the invention, the control means 6 are configured to determine the total duration (Dtot) of actual gas supply corresponding to the sum of all the time periods dt(i) between a choice, made by the user, of starting the supply of gas (e.g. start or resumption of a treatment) and a choice, made by the user, of stopping (e.g. pause or permanent cessation) the supply of gas.

The time counter is therefore incremented progressively over time as the apparatus 1 is used. The total duration (Dtot) of actual supply of gas can be expressed in days, hours and minutes, or even in weeks or months.

In other words, as illustrated in FIG. 4, the total duration (Dtot) of actual supply of gas is such that: (Dtot)=Σdt(i) with: i≥1, where i is the number of periods dt of gas use considered.

As can be seen, the start (DF) of the supply of gas to a first patient P1 commences at t0 and continues for a first duration dt1 up to t1, where a stop (AF) to the supply of gas is made due to a pause PA of the apparatus 1, during a pause time starting at t1 and continuing until t2.

At t2, the supply (DF) of gas to said first patient P1 resumes for a second duration dt2 before being permanently stopped at t3 when the healthcare staff consider that the treatment of the first patient P1 is finished or has to be stopped.

Then, the apparatus 1 is not used between t3 and t4, which is an inter-patient time period (IP), i.e. the time between the end of treatment of the first patient P1 and the start of treatment of a second patient P2.

The second patient P2 is then treated for a period of time dt3 running between t4 and t5, without a pause period in this case. The treatment of the second patient P2 is stopped at t5, which then corresponds to a new inter-patient time period (IP) preceding the treatment of a third patient.

According to the invention, the total duration (Dtot) of actual supply of gas therefore corresponds to the sum of the time periods dt1, dt2, dt3, . . . dti.

The total duration (Dtot) of actual supply of gas from the first use of the apparatus, i.e. from its first start-up, is displayed on the graphical display 7, either permanently or at the user's request, for example after pressing a dedicated reminder key or other key.

According to a preferred embodiment, the counter is never re-initialized, that is to say reset to zero (RAZ). According to another embodiment, the counter can be re-initialized, that is to say reset to zero (RAZ), after a given long period of time, for example after several months or years, or after maintenance or the like.

Advantageously, the total duration (Dtot) of actual supply of gas is permanently displayed (at 10) on the screen of the graphical display 7 so that the healthcare providers can read this off at any time.

The information concerning the duration of actual supply of gas (Dtot) can also be stored in a memory 12 of the apparatus, preferably stored in a non-volatile computer memory.

According to one embodiment, the information concerning the duration of actual supply of gas (Dtot), for example a total number of hours and/or days of treatment, can be transmitted to a hospital information system (SIH) or Patient Data Management System (PDMS). The connection between the apparatus 1 and the hospital information system can be provided by a wired link (i.e. electrical cables), typically via RS232 or HL7 connections, or by an internal network of the hospital, such as an intranet or the like, for example via a local communication protocol, in particular via WiFi or Bluetooth®. This allows the hospital to recognize the actual use of the apparatus 1 and to edit reports of medical or other care.

According to another embodiment, the information concerning the duration of actual supply of gas (Dtot) can also be transmitted by 4G to a remote server, in particular of the supplier of the apparatus and/or of the medical gases, such as NO, in order to allow the supplier to follow the “life” of the apparatus 1 and to plan maintenance operations, to schedule the replacement of the apparatus (end of legal useful life), gas refills or similar, and also to draw up invoices, usage reports, etc.

In general, the counting of the durations dt (i.e. dt1, dt2, dt3, . . . dti) of active administration by the gas administration device 1 of the invention is managed by an algorithm acting as a time counter, i.e. timer, implemented by the microprocessor of the control means 6 of the device 1 and operating as explained above.

FIG. 2 shows schematically an embodiment of a gas administration installation 20 according to the invention, in this case comprising two cylinders 21 of therapeutic gas, namely here NO/N2 mixtures, for example containing between 200 and 1000 ppm by volume of NO (remainder N2), which feed a gaseous mixture NO/N2 to a gas supply apparatus 1 according to the invention, as illustrated in FIG. 1 and described above.

The gas cylinders 21 are fluidically connected to the gas supply apparatus 1 via gas feed lines 30, such as flexible tubes or the like, which can be equipped with devices 31 for regulating and/or monitoring the gas pressure, such as a gas regulator, pressure gauges or the like. The gas feed lines 30 are connected to one or more gas inlets 3 of the gas supply device 1, which supply the internal passage 2 of said gas supply device.

It will be noted that the gas supply device 1 also comprises an oxygen inlet 33 fluidically connected, via an oxygen feed line 34 such as a flexible tube or the like, to an oxygen source (not shown), for example the hospital network, that is to say an oxygen supply duct arranged in the hospital building, or a cylinder of pressurized oxygen.

Furthermore, a medical ventilator 23 is also provided, that is to say a respiratory assistance apparatus, for supplying air or an oxygen/nitrogen mixture (N2/O2), that is to say a flow of respiratory gas containing at least 21% oxygen.

The medical ventilator 23 and the gas supply device 1 are in fluidic communication with a gas feed line 22 for conveying the gas flow to the patient.

The gas supply device 1 delivers an NO/N2 mixture, for example 800 ppmv of NO, into the gas feed line 22 via an injection conduit 37, so as to inject (at 37a) a flow of NO/N2 into the flow of air or oxygen/nitrogen mixture delivered by the medical ventilator 23 and conveyed by the gas feed line 22.

The gas feed line 22 further comprises a gas humidifier 24 arranged downstream of the site 37a where the therapeutic gas supply device 1 is fluidically connected to the feed line 22. This gas humidifier 24 humidifies the gas flow, e.g. NO/N2/air mixture, before it is inhaled by the patient by way of a patient respiratory interface, such as an endotracheal tube or the like. In FIG. 2, the patient/interface assembly is shown schematically by an “artificial lung” 25.

A line 27 for recovering the gases exhaled by the patient is also provided. The gas feed line 22 and the exhaled gas recovery line 27 are connected to a connection piece 28, preferably a Y-shaped piece, and thus define a patient circuit 29. The gas feed line 22 forms the inspiratory branch of the patient circuit 29, while the exhaled gas recovery line 27 forms the expiratory branch of the patient circuit 29.

The gas feed line 22 is fluidically connected to an outlet port 23a of the medical ventilator 23 so as to recover and convey the gas, typically air (or N2/O2 mixture containing about 21% O2) delivered by the medical ventilator 23, while the exhaled gas recovery line 27 is fluidically connected to an inlet port 23b of the medical ventilator 23 so as to supply the medical ventilator 23 with all or part of the flow of the gases exhaled by the patient.

The exhaled gas recovery line 27 can comprise one or more other optional components 26, such as a CO2 removal device, i.e. a CO2 trap, such as a hot container or the like, in order to remove the CO2 present in the gases exhaled by the patient, or a filter or the like. Indeed, a CO2 removal device can be useful when the gas contains N2O to be recovered after exhalation by the patient. In the case of NO, the exhaled gas recovery line 27 is used by the ventilator 23 to check whether there is a gas leak in the circuit 22, 27 for example.

There is also provided a flow sensor 36, for example of the hot wire or pressure differential type, connected to the gas supply device 1 via a flow measurement line 35 serving to measure the gas flow (Q′) coming from the ventilator 23, such as air (i.e. N2/O2), within the feed line 22, upstream of the connection and NO/N2/air mixing site (at 37a). As has been explained above, this makes it possible in particular to regulate the passage of the NO through the solenoid valves 5 of the device 1.

In addition, a gas sampling line 38 can be provided which fluidically connects the gas supply device 1 to the feed line 22, close to the Y-piece 28, serving to take gas samples and to verify their compliance with the desired mixture to be administered to the patient. The sampling line 38 is connected to the conduit 22, i.e. the inspiratory branch of the patient circuit 29, downstream (at 38a) of the connection site 37a of the injection conduit 37, as seen in the direction of the gas flow from the ventilator 23 to the patient.

The final gas flow (i.e. NO/N2/O2 mixture) resulting from the mixing of the air flow from the ventilator 23 and the NO/N2 flow from the apparatus 1 is supplied to the patient via a respiratory interface, such as a breathing mask or endotracheal intubation tube, which are shown schematically here by an “artificial lung” 25.

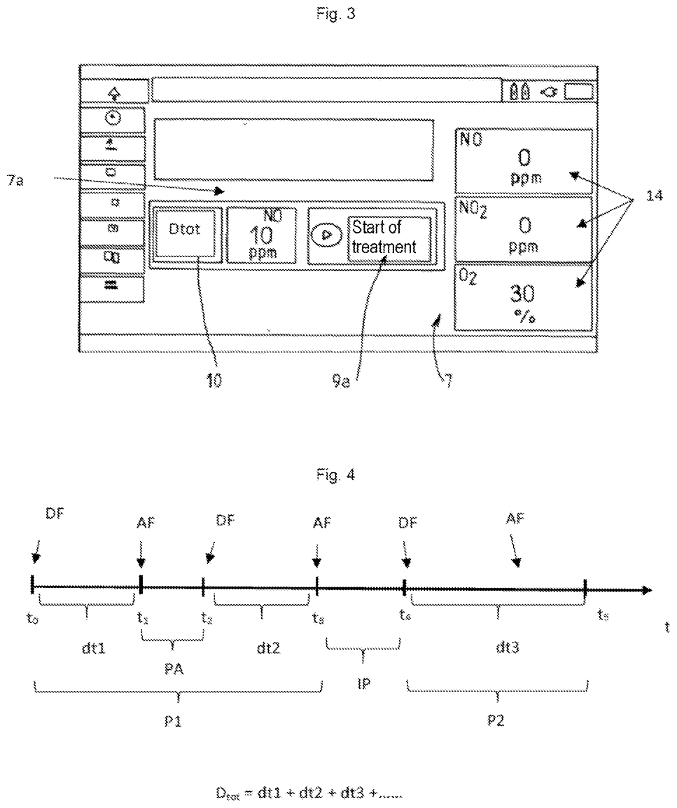

FIG. 3 shows schematically an embodiment of the screen of the graphical display 7 of a gas supply apparatus 1 according to the invention, which is configured to display various items of information, such as the concentrations of NO, O2 and NO2 (at 14) in the gas, but also, according to the invention, a single touch key (e.g. a window or the like) 9a for “Start” and “End” of the supply of gas (e.g. NO), allowing the user, i.e. a member of the healthcare staff, to start or to temporarily or definitively stop a treatment by administration of gas, such as NO, to a patient.

As has been explained above, the screen of the graphical display 7 shows the total duration Dtot (at 10) of actual supply of gas to the one or more patients who have been treated with this apparatus 1.

Since the problem of recording the treatment time of patients is common in public and private hospitals, the device of the invention can be used for treatments by gas other than that by inhaled nitric oxide, i.e. iNO, for example for treatments based on an equimolar mixture of oxygen and nitrous oxide, such as MEOPA, or another gas or gaseous mixture (e.g. argon/O2, He/O2O2, etc.).

According to one embodiment, the control means 6, in particular the time counter integrated into said control means 6, are also configured to determine, i.e. to measure, the instantaneous duration (Dinst) of an ongoing treatment of a given specific patient, that is to say the total treatment time of this specific patient, in particular treatment by NO, and to control a display, on the graphical display 7, of this instantaneous duration (Dinst) corresponding to the treatment time of the patient in progress, in particular during treatment by administration of NO.

This count and the display of the instantaneous duration (Dinst) relating to an ‘ongoing treatment’ are reset to zero when a healthcare provider initiates a start of a new treatment, by clicking on a treatment “Start” key or a “New Patient” key acting as reset (RAZ) or re-initialization means, i.e. reset key, used to reset the display of the instantaneous duration (Dinst) on the graphical display at the end of the treatment of a given patient and before the start of treatment of a following patient.

This instantaneous duration (Dinst) of an ongoing treatment of a given specific patient can also be used to determine whether this treatment is a short treatment TC or a long treatment TL, and therefore the number of short treatments (TC) for which the total duration of treatment is less than a predetermined threshold duration and their respective durations (DTC), and/or, conversely, the number of long treatments (TL) for which the total duration of treatment is greater than the predetermined threshold duration and their respective durations (DTL), preferably a predetermined threshold duration less than or equal to 6 hours. Advantageously, a short treatment TC also has a minimum duration, i.e. low threshold, of at least 10 min.

In general, the invention also relates to the use, for treatment of a patient suffering from a pulmonary disease, of an installation 20 for administering gas to a patient, comprising one or more gas sources 21 containing an NO/N2 mixture, comprising the gas supply apparatus 1 according to the invention, fed with gas by the one or more therapeutic gas sources 21, and a medical ventilator 23 delivering a gas stream containing at least 20% oxygen, such as air or an O2/N2 mixture, which are fluidically connected to a gas feed line 22 for mixing therein the NO/N2 mixture with the gas stream containing at least 20% oxygen and for obtaining an NO/N2/O2 mixture containing mainly NO, nitrogen and oxygen, which is then administered by inhalation to the patient to be treated. The nitric oxide (NO) present in the NO/N2/O2 mixture inhaled by the patient is a gaseous medicament permitting treatment of acute pulmonary arterial hypertension, particularly pulmonary vasoconstrictions in adults or children, including the newborn, such as persistent pulmonary hypertension of the neonate (PPHN), or in the context of cardiac surgery with extracorporeal blood circulation (CEC) that may cause pulmonary vasoconstrictions.

An NO administration installation of this kind is mainly intended for use in a hospital environment for administering treatment by NOi and thus for treating hospital patients who need to inhale NO in order to treat their pulmonary arterial hypertension.

What is claimed is:

1. Gas supply apparatus (1) comprising:
at least one internal passage (2) for supplying a flow of gas, comprising valve means (5),
control means (6) with microprocessor, instructing the valve means (5) to control the supply of gas, and
selection means (8) which can be actuated by a user and are configured to allow the user to start or stop a supply of gas,
characterized in that the control means (6) are configured to count a total duration (Dtot) of actual supply of gas by the apparatus (1) corresponding to the cumulative total of all the time periods (dti) that have elapsed between a start and a stop of a supply of gas by the apparatus.

2. Apparatus according to claim 1, characterized in that:
it further comprises selection means (8) which can be actuated by a user and are configured to allow the user to choose between starting a supply of gas and stopping a supply of gas, and
the total duration (Dtot) of actual supply of gas by the apparatus (1) corresponds to the cumulative total of all the time periods (dti) that have elapsed between a choice, made by the user, of starting a supply of gas (DF) and a choice, made by the user, of stopping a supply of gas (AF), by the user actuating the selection means (8) of the apparatus (1).

3. Apparatus according to claim 1, characterized in that it comprises a graphical display (7) controlled by the control means (6), or the selection means (8) comprise one or more selection keys (9a-9d).

4. Apparatus according to claim 3, characterized in that the graphical display (7) comprises a touch screen (7a), and the one or more selection keys (9a-9d) are virtual keys displayed on the touch screen (7a).

5. Apparatus according to claim 1, characterized in that the control means (6) are configured to control a display, on the graphical display (7), of the total duration (Dtot) of actual supply of gas.

6. Apparatus according to claim 1, characterized in that:
the selection means (8) cooperate with the control means (6) to supply them with at least one command signal corresponding to the choice made by the user, and
the control means (6) instruct the valve means (5) to control the flow of gas in said at least one internal passage (2), in response to the command signal supplied by the selection means (8), so as to authorize or stop any flow of gas.

7. Apparatus according to claim 1, characterized in that the control means (6) comprise a time counter integrated in the control means (6), in particular in the microprocessor, in particular an algorithm acting as a time counter.

8. Apparatus according to claim 7, characterized in that the time counter integrated in the control means (6) is configured to count all the time periods (dti) elapsing between a choice, made by the user, of starting a supply of gas (DF) and a choice, made by the user, of stopping a supply of gas (AF), excluding any period of non-supply of gas (PA, IP).

9. Apparatus according to claim 1, characterized in that it comprises storage means for storing the total duration (Dtot) of actual supply of gas and/or all the time periods (dti).

10. Apparatus according to claim 1, characterized in that the control means (6) are further configured to determine an instantaneous duration (Dinst) of an ongoing treatment, and the graphical display (7) is configured to display said instantaneous duration (Dinst).

11. Apparatus according to claim 1, characterized in that the control means (6) are configured to determine a short treatment (TC) or a long treatment (TL) from the instantaneous duration (Dinst) of an ongoing treatment of a given patient.

12. Installation (20) for administering gas to a patient, comprising:
at least one source (21) of gas, such as an NO/N2 mixture,
a gas supply apparatus (1) according to one of the preceding claims, fed with gas by said at least one therapeutic gas source (21), and
a gas feed line (22) fed with gas by the gas supply apparatus (1).

13. Installation according to claim 12, characterized in that it further comprises a medical ventilator (23) in fluidic communication with the gas feed line (22).

14. Installation according to claim 12, characterized in that said at least one gas source (21) contains an NO/N2 mixture.

15. Installation according to claim 12, characterized in that the gas source contains an NO/N2 mixture containing less than 2000 ppm by volume of NO, the remainder being nitrogen, preferably less than 1000 ppm by volume of NO, the remainder being nitrogen.
